(19) United States
(12) Patent Application Publication
(10) Pub. No.: US 2003/0077825 A1
(43) Pub. Date: Apr. 24, 2003

(54) Structures useful for bone engineering and methods

(76) Inventors: Rajendra S. Bhatnagar, Burlingame, CA (US); Jing Jing Qian, Foster City, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/133,289

(22) Filed: Apr. 25, 2002

Related U.S. Application Data
(63) Continuation of application No. 09/561,554, filed on Apr. 28, 2000, now abandoned.
(63) Continuation-in-part of application No. 09/816,737, filed on Mar. 23, 2001.
Application No. 09/816,737 is a continuation of application No. 09/328,347, filed on Jun. 8, 1999, now Pat. No. 6,268,348.
Application No. 09/328,347 is a continuation of application No. 08/859,610, filed on May 20, 1997, now Pat. No. 5,958,428.
Application No. 08/859,610 is a division of application No. 08/278,878, filed on Jul. 22, 1994, now Pat. No. 5,635,482.

(51) Int. Cl. A61F 2/28

(52) U.S. Cl. 435/395; 623/23.63; 623/919

(57) ABSTRACT

A bone repair apparatus is provided where a biologically compatible structure has a compound carried on the structure that mimics collagen binding to cells. Living cells derived from fibroblasts are carried on the structure and display at least one morphologic change consistent with an osteogenic phenotype. The preferred method for practicing the invention includes harvesting a quantity of fibroblasts from a patient in need of a bone graft, growing the tissue under cell growth conditions, and seeding at least some cells of the cultured tissue on the biologically compatible structure with the collagen mimic thereon. The culture tissue cells are seeded on the structure and incubated under cell growth conditions, which results in the differentiation of the cells to bone-like cells and thus provides a tissue engineered apparatus ready for use as a bone graft.

This invention was made with government support under Grant No. DE 11619, awarded by the National Institutes of Health. The Government has certain rights in this invention.

### FIELD OF THE INVENTION

The present invention generally relates to tissue engineering applications, and more particularly for repair and treatment of bony defects by uses of structures carrying a collagen mimic and seeded with fibroblasts. These fibroblasts differentiate on the structures so as to become bone-like cells. Structures of the invention with such trans-differentiated cells are useful as living bone grafts. When implanted, they integrate with host bone and repopulate host bony sites lacking viable bone cells because of disease or radiation therapy.

### BACKGROUND OF THE INVENTION

Next to blood, bone is the most transplanted human tissue. Approximately 1.3 million surgical procedures involving bone grafts, bone replacement, augmentation, or other reconstructive operations are carried out every year in the United States alone. Currently over 90% of the living grafts are autografts, requiring additional trauma and morbidity to the patient. By contrast to autografts, allografts carry the inherent risk of pathogen transfer, and require long term immunosuppression. Thus there is a major need for a procedure that will provide living bone tissue for grafts which may be prepared, preferably from a patient's own cells, or less preferably from pathogen-screened donor cells.

Recent advances in the fields of cell and molecular biology, biotechnology, and biomaterials have led to the emergence of tissue engineering, an exciting new discipline applying both engineering and life science principles to the formation of biological substrates capable of regenerating functional mammalian tissues both in vitro and in vivo. Present attempts involved the isolation of cells from biopsies of existing tissue which were seeded onto three-dimensional carrier materials. A major limitation of these techniques is that the cells are often procured from an autologous source. Alternatively, the procurement of cells from cadavers carries the inherent risk of transfer of pathogens, and the undue expense of screening for presence of harmful pathological agents. The drawback of both of these approaches stems from the source of cellular material.

It is known that connective-tissue cells, including fibroblasts, cartilage cells, and bone cells, can undergo radical changes of character. Thus, as explained by Alberts et al., Molecular Biology of the Cell, (2^{nd} Ed., 1989, pp. 987-988), a preparation of bone matrix may be implanted in the dermal layer of the skin and some of the cells there are converted into cartilage cells and others into bone cells.

U.S. Pat. No. 5,700,289, issued Dec. 23, 1997, inventors Breitbeart et al., describe the use of periosteal cells that are seeded onto a matrix for implantation and then cultured under conditions which are said to induce the cells to form bone rather than other tissue types. However, the use of periosteal cells to seed implants entails invasive surgery.

Autologous grafts of fresh bone yield the best results of current procedures because they contain living differentiated cells already in their physiologic environment; however, autografts require additional trauma as part of harvest procedures. Preserved autografts and freeze-dried bone allografts are also effective because they are likely to possess appropriate physico-chemical and mechanical properties. These materials are less effective than living bone, because they lack living cells, and because their physical state may not permit penetration by cells. De-mineralized bone preparations contain extracellular matrix and other organic components of bone, and this may explain the minor improvement observed in some patients. Despite claims that freeze drying may reduce antigenicity, the potential risk of disease transmission by pathogen transfer remains a major concern in the use of organic bone products. However, the use of variety of natural and synthetic minerals, polymers and composites without the presence of suitable, living cells seed thereon has not provided satisfactory bone restoration and often there is only a fibrotic response.

A variety of biologically compatible materials are known. Some, for example, are discussed by Hollinger et al., “Biodegradable Bone Repair Materials,” Clinical Orthopedics and Related Research, 207, pp. 209-305 (1986), and Elgendy et al., “Osteoblast-Like Cell (MC3T3-E1) Proliferation on Bioerodable Polymers: An Approach Towards the Development of Bone-Bioerodable Polymer Composite Material,” Biomaterial, 14, pp. 263-269 (1993). For other examples, materials such as collagen gels, poly(lactide) [PLA] and poly(lactide-co-glycolide) [PLGA] fiber matrices, polyglactin fibers, calcium alginate gels, polyglycolic acid (PGA meshes, and other polyesters such as poly-(L-lactic acid) [PLLA], and polyanhydrides are among those suggested and are in varying degrees of development and use. Thus, a great variety of materials, including natural and synthetic polymers, are known and useful as scaffolds, templates, or matrices for preparing implants.

Another class of materials suitable for implant are ceramics, such as hydroxyapatite, or similar ceramics formed of tricalcium phosphate or calcium phosphate.

Repair compositions comprising hydroxyapatite particles admixed with a quantitative of synthetic peptide, where the peptide has a domain that mimics collagen binding, are described by U.S. Pat. No. 5,354,736, issued Oct. 11, 1994, inventor Bhatnagar. Implants comprising a matrix formed of a biomaterial and a peptide carried by the matrix and having enhanced cell binding with respect to collagen are described by U.S. Pat. No. 5,636,482, issued Jun. 3, 1997, inventor Bhatnagar. U.S. Pat. No. 5,661,127, issued Aug. 26, 1997, and U.S. Pat. No. 5,780,436, issued Jul. 14, 1998, both having inventors Bhatnagar and Qian, disclose tissue repair compositions that comprise a biocompatible matrix having small peptide mimics of TGF-β that are admixed with or carried by the matrix.

### SUMMARY OF THE INVENTION

In one aspect of the present invention, a method for preparing a bone repair apparatus is provided which comprises the steps of: (i) harvesting a quantity of fibroblasts, such as dermal, gingival, or periodontal tissue, from a patient in need of a bone graft, (ii) growing the tissue under cell growth conditions to form cultured tissue cells, and (iii) seeding at least some of the cultured tissue cells on a biologically compatible structure, wherein the seeded cells differentiate into an osteogenic phenotype when incubated under cell growth conditions. The biologically compatible structure that is provided for practicing the inventive method includes a collagen mimic. When cultured cells have been seeded on the biologically compatible structure, they are exposed to the structure and its collagen mimicking compound, and they differentiate into an osteogenic phenotype.

Consequently, in practicing the bone tissue method, fibroblast cells from the recipient can be easily harvested with minimal invasion and trauma to the patient. Bone cells/osteoblasts are scarce and harvesting them causes trauma. By contrast, fibroblasts are plentiful and easily obtained with minimal trauma and by practicing the inventive method one is able to obtain living bone grafts. The easily harvested fibroblasts are converted to living bone-like cells and they, together with the biologically compatible structure, yield a tissue-engineered bone graft. This can integrate with host bone when implanted in the patient, and repopulates host sites lacking viable cells because of disease or radiation therapy.

In another aspect of the present invention, a method for treating injured or diseased bone in a patient, comprises the step of: (i) providing a bone repair apparatus having a biologically compatible structure with a compound carried on the structure, the compound having a domain mimicking collagen binding to cells but having enhanced cell binding with respect to collagen. The bone repair apparatus further has living cells growing on the structure that display at least one osteogenic marker. These living cells have been derived from fibroblasts, preferably taken from the patient being treated, and have differentiated into an osteogenic phenotype wherein they display at least one osteogenic marker. Preferred embodiments for the structure include a ceramic, such as hydroxyapatite. The treatment method further includes the step of: (ii) implanting the bone repair apparatus in the patient from whom the cells displaying the osteogenic marker were derived. The implant will generate bone-like tissue in vivo.

### BRIEF DESCRIPTION OF THE DRAWINGS

FIG. 1 schematically illustrates preparation of inventive embodiments and practice of the invention;

FIG. 2 graphically illustrates the relationship between concentration of a peptide embodiment of the invention on the horizontal axis with respect to the amount of the peptide absorbed therefrom on a structure in accordance with the invention;

FIG. 3 graphically illustrates that fibroblast cells were bound to the inventive structure embodiment as a dependent function of the peptide concentration of the structure;

FIGS. 4(A) and 4(B) are light micrographs of two different parts of histological sections (400× magnification) illustrating the osteoblast-like morphology (A) of the original fibroblast cells during Step 16 of FIG. 1; and,

FIG. 5, Panels (A) and (B), illustrates total RNA Northern Blot analysis for four osteogenic phenotype markers after 7, 11, and 14 days in culture, with the control human gingival fibroblasts (on a structure not including an inventive peptide) shown on the left (A) while human gingival fibroblasts growing on an inventive embodiment are shown on the right (B), and illustrate the electrophoresis of a reverse transcriptase PCR.

### DETAILED DESCRIPTION OF THE INVENTION AND PREFERRED EMBODIMENTS

Engineered tissue analogs require an ample supply of cells, a hospitable template with desirable biomechanical characteristics, and conditions that would facilitate differentiation. Tissue implant embodiments for practicing the present invention include a biologically compatible structure, which serves as template, scaffold, or matrix. These terms shall hereinafter be included in the single, broad term “structure.” The structure carries a collagen mimic. This collagen mimic compound has a domain that mimics collagen binding to cells, but has enhanced cell-binding with respect to collagen. The collagen mimic compound has been found to facilitate differentiation of fibroblasts to cells with an osteogenic phenotype. Particularly preferred embodiments for the suitable biologically compatible structures are described by U.S. Pat. No. 5,958,428, issued Sep. 28, 1999, inventor Bhatnagar, incorporated herein by reference. As described in that patent, there is a family of synthetic peptides that mimic the cell binding domain of collagen, but which have enhanced cell binding with respect to collagen. One preferred member of that family is sometimes called “P-15” (and is referred to as “peptide (1)” in some of the figures). This P-15 synthetic peptide has the amino acid sequence Gly-Thr-Pro-Gly-Pro-Gln-Gly-Ile-Ala-Gly-Gln-Arg-Gly-Val-Val (SEQ ID NO: 1), and which fifteen amino acid embodiment has the same sequence as a particular, small region in the α1(I) chain of collagen.

However, it is the central portion, forming a core sequence, of the P-15 region that is essential for the desired collagen-like activity. Thus, peptides for use in this invention may be of a different length than P-15 and even may have amino acid variations from P-15 but such peptides will typically contain the sequence Gly-Ile-Ala-Gly (SEQ ID NO:2). The two glycine residues flanking the fold, or hinge, formed by -Ile-Ala- are hydrogen bonded at physiologic conditions and thus stabilize the β-fold. Because the stabilizing hydrogen bond between glycines is easily hydrolyzed, two additional residues flanking this sequence can markedly improve the cell binding activity by further stabilizing the bend conformation.

Useful compounds to be carried on the structure with the desired collagen-like properties should be selected on the basis of similar spacial and electronic properties, as compared to P-15 or to a portion thereof in view of the β-fold stabilization feature discussed above. These compounds typically will be small molecules of 100 or fewer amino acids or in the molecular weight range of up to about 5,000 daltons, more typically up to 2,500 daltons. Nonpeptides mimicking the necessary β-fold conformation so as to have recognition and docking of collagen binding species are also contemplated as being within the scope of this invention.

Suitable biologically compatible structures (that is, biomaterials) include the materials earlier described as being known for use as scaffolds, or matrices, in implant applications. For example, collagen gels, PLG fiber matrices, and PGA meshes are suitable and have been earlier noted as known to the art. However, particularly preferred is the use of natural or synthetic hydroxyapatite, calcium phosphates, or combinations of these with other inorganic or organic materials, polymeric carriers, or binders.

The collagen mimic may be carried on the structure by any of a variety of methods and modes of attachment. The structures may be made with the necessary collagen mimic adsorbed or bonded to structure surfaces, whether composed of porous or nonporous materials, such as those containing anorganic bone mineral (ABM), natural or synthetic hydroxyapatites, calcium phosphates, and other inorganic or organic compositions. These structures may be featured into desired shapes or blocks that may be machined to obtain specific shapes for particular applications. When the collagen mimic is desired to be covalently bonded to the structure, then well-known methods for forming covalent linkages may be used. Non-covalent interactions and non-specific adsorption typically involve the direct application of a solution, preferably a saturated solution, containing the suitable collagen mimic to the structure.

In preparing implant embodiments of the invention, one preferably takes fibroblast tissue from a patient in need of bony tissue repair. The autograft may then be used directly from the recipient to prepare transplants in accordance with the invention, or the fibroblasts of the recipient may be stored when the procedures for grafting are anticipated in the future. Allografts of living bone constructs can also be prepared from fresh or stored donor fibroblasts matched for compatibility with the recipient. Alternatively, donor fibroblasts may be used in conjunction with immunosuppression.

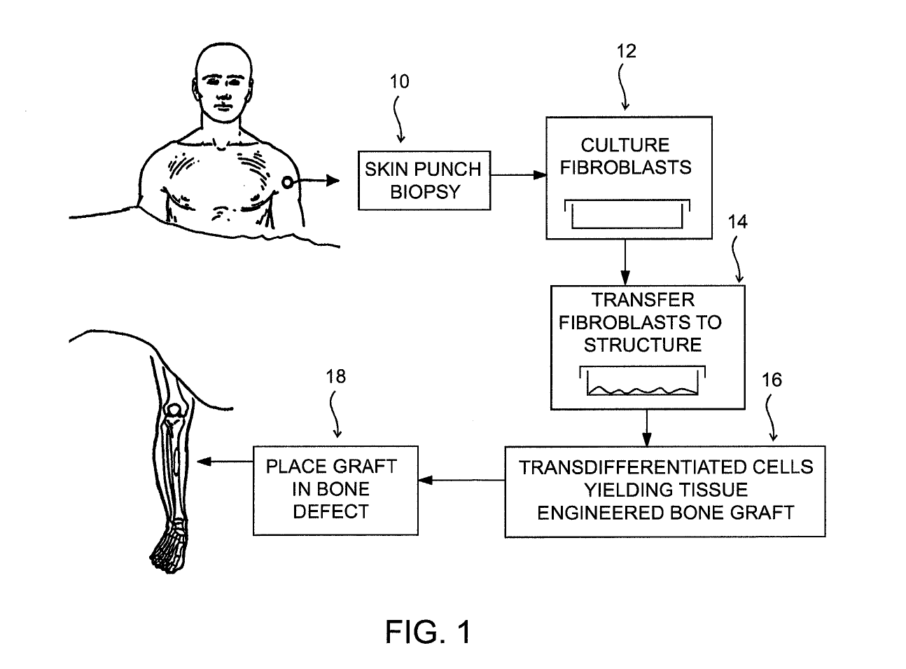

Turning to FIG. 1, preparation of inventive implant embodiments is schematically illustrated. Thus, Step 10 illustrates obtaining fibroblasts from a patient in need of bony tissue repair, such as by skin punch biopsy. These fibroblasts are then cultured in Step 12 by standard cell culture methods and in medium well known to the art. The fibroblast culture is preferably, though it need not be, brought to confluence.

Meanwhile, structures are prepared to receive the cultured fibroblasts. These structures as described above carry the necessary collagen mimic, such as wherein the collagen mimic is adsorbed or bonded to surfaces of porous or nonporous material. This is graphically illustrated by Step 14 of FIG. 1. Thus, in preparing preferred embodiment implants, fibroblasts derived from species such as human dermis, human gingival, and human periodontal ligament, respectively, have been seeded onto structures composed of ABM to which P-15 was adsorbed.

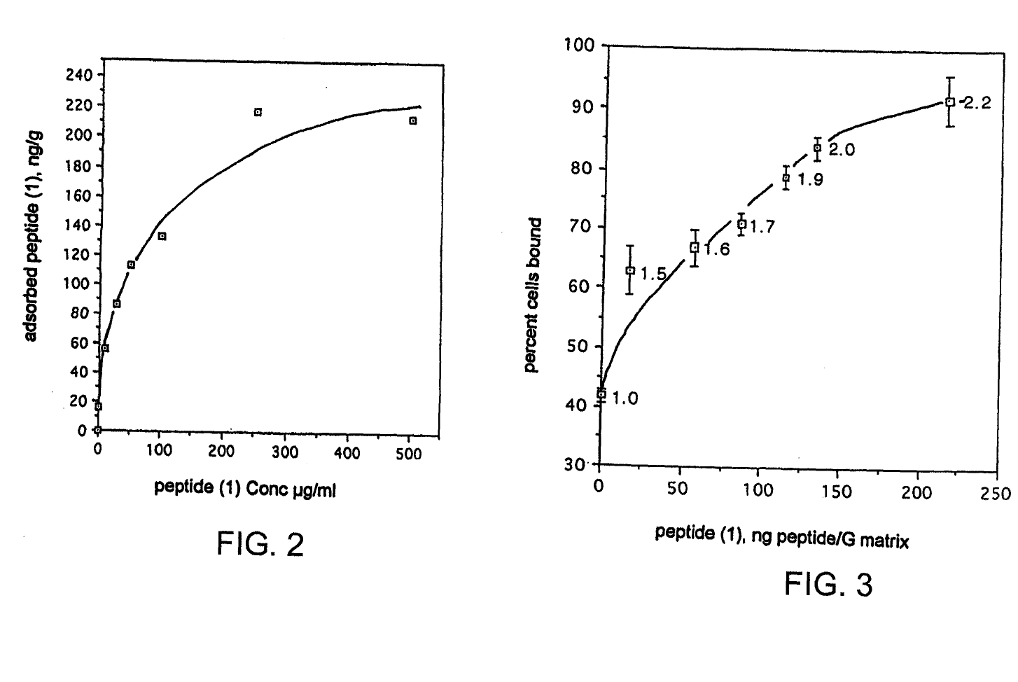

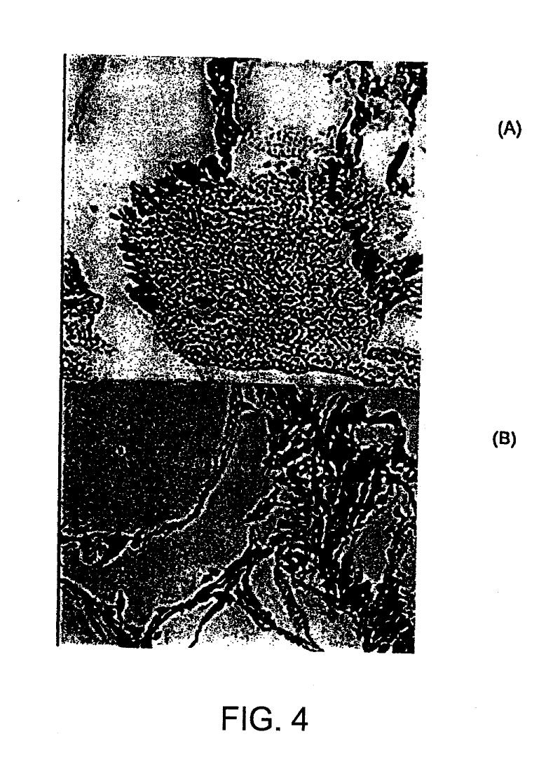

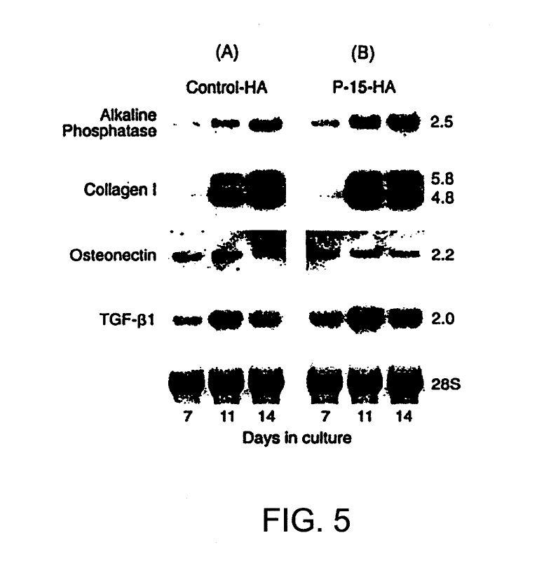

Turning to FIG. 2, one can see that exposing the structure (ABM) to increasing amounts of the collagen mimic peptide (P15) in solution resulted in increasing adsorption of the peptide on the structure. In subsequent experiments illustrating the invention (FIGS. 4 and 5), the peptide containing solutions to which the ABM was exposed were fully saturated with the peptide.

Turning to FIG. 3, the inventive structure (ABM) carrying adsorbed peptide (P15) was then tested for binding of fibroblasts. In the experiments illustrated by FIG. 3, radiolabeled human periodontal ligament fibroblasts were incubated for 24 hours with structures containing varying amounts of the peptide. As is shown by the FIG. 3 data, the number of cells bound to the structures increased with the increasing peptide content. The numbers next to each data point in the graph are the ratio of cells bound to the structures at each peptide concentration with respect to cells bound to a control structures carrying no collagen mimic peptide.

These implant embodiments have resulted in the fibroblast cells forming three-dimensional colonies and displaying major morphologic changes consistent with an osteogenic genotype. Thus, turning to FIG. 4, Panels (A) and (B) illustrate human dermal fibroblasts cultured on an embodiment of the invention (wherein there was 160 ng peptide/gram, which was a saturating concentration at which more than 90% cells are bound). These fibroblasts are observed to form three-dimensional colonies that are characterized by osteoblast-like morphologies. This is illustrated in the schematic of FIG. 1 by Step 16 such that the fibroblasts are transdifferentiating.

The markers of osteogenic differentiation include the expression of messenger RNAs for type I collagen, collagenase (MMP-1), alkaline phosphatase, osteonectin, and TGF-β. These markers are highly characteristic of the osteogenic phenotype. This is illustrated by FIG. 5. In FIG. 5 human gingival fibroblasts were cultured either on a control ABM structure (with no peptide) or an inventive embodiment structure (analogous to that described for FIG. 4). Panel (A) of FIG. 5 shows the fractions by gel electrophoresis after 7, 11, and 14 days in culture, and similarly Panel (B) illustrates the fractionated RNA molecules but when cultured on the inventive embodiment. As illustrated by Panel (B), the cultures on the inventive embodiment displayed increasing expression of bone markers, including alkaline phosphatase, type I collagen, and TGF-β-1. The decreasing expression of osteonectin is also consistent with osteo-differentiation over these periods of time.

Returning to FIG. 1, step 18 illustrates placing, that is implanting, the inventive embodiment in the bone defect of the patient from whom the fibroblasts had originally been taken.

The following experimental work is intended to illustrate, but not limit, the invention.

### EXAMPLE 1

Peptide Synthesis: The peptide P-15, GTPGPQGIAGQRVV (SEQ ID NO: 1), was synthesized by solid phase procedures using 9-fluorenylmethoxycarbonyl protecting groups, except for glutamine residues which were coupled with 1-hydroxybenzyl triazole. The peptide was purified to by reverse phase HPLC using a C-18 column in a gradient of H₂O and acetonitrile. The purity of the peptide used was >95%. The amino acid sequence was confirmed by sequence analysis.

Structure Material: Bovine bone derived porous ABM (anorganic bovine bone mineral) in a particulate form with a particle size of 250-420 μm was obtained from a commercial source. The ABM had a mean pore volume of 0.13 cc/g and a total porosity of 28% based on mercury porosimetery. The manufacturer had certified that deproteination was complete based on Kjeldahl and carbon analyses and the purity was further warranted by x-ray diffraction standard. Microanalytical procedures used in our laboratory confirmed the absence of nitrogenous materials in the ABM preparation.

Preparation of Structure with Collagen Mimic: P-15 was adsorbed on ABM in a saturable manner, and the binding of cells to ABM-P-15 was proportional to the amount of adsorbed P-15. The complex between ABM and P-15 was stable under physiological conditions. The peptide was adsorbed on ABM by incubating the particulate mineral for 24 hours at 20° in a solution of the peptide in phosphate buffered saline (PBS) in a ratio of 1.0 g:2.0 ml solution containing 100 μg/ml of the peptide. Following incubation, ABM was washed three times by shaking with 5× volume of PBS over a 24 hour period to remove unadsorbed peptide. The ABM-P-15 powder was collected and dried in a desiccator over Drierite. The preparations were sterilized by γ-irradiation. Analyses for adsorbed peptide were made using these sterilized preparations. The peptide content was assayed by amino acid analysis of 2.0 N NaOH hydrolysates. FIG. 2 shows the adsorption of different amounts of P-15 to ABM as a result of incubation with different concentrations of P-15 in PBS.

Tissue Engineering on the Collagen-Mimic Structure: Three-dimensional culture was carried out on the ABM-P-15 particulate support placed in silicone-treated glass dishes. Binding of cells to ABM-P-15 is proportional to the amount of P-15 present in the BM-P-15 composite (FIG. 3). Cells bound to ABM-P-15 formed three-dimensional colonies (FIGS. 4(A) and 4(B)), which is a requirement for differentiation and tissue engineering. The cultured cells were incubated on the structures under standard cell growth conditions for about seven or more days, at which time they had differentiated into bone-like cells.

Histology: The samples were fixed for histological examination. Sections were cut with a low speed diamond saw, in different planes in order to obtain maximum information concerning the ingrowth of bone-like cells. Histochemical procedures were used to examine the synthesis and deposition of major components of bone and to investigate the secretion of alkaline phosphatase, a marker of osteogenic differentation.

Staining with Alizarin Red and Von Kossa for Mineralization: Positive staining with Alizarin Red or Von Kossa stains is an indicator of mineralization. Cultures on ABM and ABM-P-15 were prepared for staining by removing the medium, washing the cells with phosphate buffered saline, and fixing in 80% ethanol for five minutes following which the cells were gently washed with H₂O. The cells were stained and excess stain removed by destaining in 95% ethanol +5% concentrated HCl for ten minutes. The samples were air dried, examined, and photographed in a light microscope. Inspection of the samples showed the formation of Alizarin reactive material in living bone graft embodiments made with dermal, gingival, and periodontal ligament fibroblasts. Thus, at 7 days and 14 days after culture on ABM, only faint, background staining was seen. In contrast, dermal fibroblast cultures on the inventive structure displayed the deposition of new bone-like matrix characterized by intense red staining after 7 days and 14 days. This confirmed the deposition of new bone matrix.

Electron Microscopy: Samples were fixed in 2.5% glutaraldehyde in sodium cacodylate buffer, and in osmium tetroxide, and dehydrated in a graded series of ethanol. Before preparation of scanning electron micrographs, the samples were subjected to critical point drying and coating with gold/palladium. SEMs were prepared in a Philips XL40 scanning electron microscope.

Molecular Biological Markers for Osteogenic Differentiation: The expression of messenger RNAs for type I collagen (MMP-1), alkaline phosphatase, osteonectin, and TGF-β were examined as criteria for osteogenic differentiation. These markers are highly characteristic of the osteogenic phenotype. Gene expression for living tissue made with gingival fibroblasts by practicing the invention is shown in FIG. 5(B).

### EXAMPLE 2

Induction of osteogenic marks in human dermal fibroblasts was also confirmed by examining the expression of bone-related genes (i) type I collagen, (ii) alkaline phosphatase, (iii) bone morphogenic protein-2 (BMP-2), and (iv) bone morphogenic protein-4 (BMP-4) using the Reverse Transcriptase-Polymerase Chain Reaction (RT-PCR).

RT-PCR was carried out on total RNA extracted by TRIZOL reagent (Gibco BRL) from the cultures at different times as indicated below. Briefly, the RNA to cDNA product was achieved using the SUPERSCRIPT pre-amplification system (BRL) in RT-PCR. The RT-PCR reaction was carried out with forward and downward primers, in a total volume of 50 μl containing buffer (Tris.IICl 67 mM, pII 8.8; (NH₄)₂SO₄ 16.6 mM; Triton X-100, 0.45%; and gelatin, 0.2 mg/ml); MgCl₂ 25 mM; dNTP mixture, 200 μM; primers 0.2 μM; SUPERSCRIPT transcriptase (BRL), 1.0 unit; Taq polymerase (BRL), 1.0 unit; and 10-200 ng mRNA. The following PCR parameters were used:

Cycle 1

94° C., 2 min

55° C., 1 min

72° C., 1 min

followed by 30 cycles

94° C., 30 sec

60° C., 40 sec

72° C., 90 sec

and then

72° C., 7 min

The product was stored at 4° until analysis by electrophoresis. A DNA ladder was used to identify the product.

The results of these experiments are presented in FIG. 2. As seen in FIG. 2, there is a marked increase in the expression of type I collagen in dermal fibroblasts growing on ABM-P-15 matrices. Marked stimulation of alkaline phosphatase gene expression in HA-P-15 cultures is consistent with the induction of a bone-like phenotype.

It is to be understood that while the invention has been described above in conjunction with preferred specific embodiments, the description and examples are intended to illustrate and not limit the scope of the invention, which is defined by the scope of the appended claims.

It is claimed:

1. A method for preparing a bone repair apparatus, comprising:
harvesting a quantity of fibroblasts from a patient in need of a bone graft;
growing the fibroblasts under cell growth conditions to form cultured tissue cells;
providing a biologically compatible structure having a collagen mimic carried on the structure, the collagen mimic having enhanced cell-binding with respect to collagen; and,
seeding at least some of the cultured tissue cells on the provided structure, wherein the seeded cells are in the presence of the collagen mimic carried thereon, and incubating the seeded cells under cell growth conditions, wherein the seeded cells differentiate into an osteogenic phenotype.

2. The method as in claim 1 wherein the fibroblasts are dermal, gingival, or periodontal tissue.

3. The method as in claim 1 wherein the incubating is in vitro.

4. The method as in claim 1 wherein the differentiation of seeded cells is determinable by at least one osteogenic differentiation marker.

5. The method as in claim 4 wherein the seeded cells display increasing RNA expression of alkaline phosphatase, type I collagen, and TGF-β-1.

6. The method as in claim 1 wherein the structure includes natural or synthetic hydroxyapatite, anorganic bone mineral, and/or calcium phosphates.

7. The method as in claim 1 wherein the structure has the SEQ ID NO: 1 peptide adsorbed thereon or bonded thereto.

8. The method as in claim 7 wherein a saturated solution of the peptide was adsorbed on the structure.

9. A method of repairing a bony defect in a patient, comprising:
providing the bone repair apparatus of claim 1; and, implanting said apparatus in the patient at the bony defect.


SEQUENCE LISTING (2 sequences)

SEQ ID NO: 1 - protein, 15 aa, Artificial Sequence
  Synthetic Peptide
GTPGPQGIAGQRGVV

SEQ ID NO: 2 - protein, 4 aa, Artificial Sequence
  Synthetic Peptide
GIAG